(19) United States
(12) Patent Application Publication
(10) Pub. No.: US 2024/0158859 A1
(43) Pub. Date: May 16, 2024

(54) METHODS OF DETECTING CONGENITAL HEART DISEASE

(71) Applicant: City University of Hong Kong, Hong Kong (HK)

(72) Inventors: Shuaicheng LI, Hong Kong (HK); Wenqian ZHANG, Hong Kong (HK)

(21) Appl. No.: 18/501,063

(22) Filed: Nov. 3, 2023

Related U.S. Application Data
(60) Provisional application No. 63/425,667, filed on Nov. 15, 2022.

(51) Int. Cl. C12Q 1/6883

(52) U.S. Cl. CPC C12Q 1/6883; C12Q 2600/118; C12Q 2600/156; C12Q 2600/158

(57) ABSTRACT

A method for detecting congenital heart disease (CHD) in a fetus is provided. Particularly, specific DNA structural variants (SVs) are identified as reliable indicators of CHD. The method begins with the collection of a maternal blood sample. Within this blood sample lies the pivotal source of fetal genomic DNA, a genetic information about the fetus. This genomic DNA is subjected to sequencing analysis for specific CHD-susceptible structural variants, including head-to-tail deletions or tail-to-head duplications occurring on chromosomes 5, 6, or 15. The measured fetal genomic DNA is pitted against a database created by the analysis of control subjects who bear no CHD, employing whole-genome sequencing and threshold values. When the measured genomic DNA exceeds these thresholds, it is indicated that a pregnant woman stands at an elevated risk of bearing a fetus with CHD, paving the way for early intervention and care.

### CROSS-REFERENCE TO RELATED APPLICATIONS

The present application claims priority from U.S. provisional patent application Ser. No. 63/425,667 filed Nov. 15, 2022, and the disclosure of which is incorporated herein by reference in its entirety.

### FIELD OF THE INVENTION

The present invention generally relates to the medical field. More specifically, the present invention relates to the techniques for screening congenital heart diseases.

### BACKGROUND OF THE INVENTION

Congenital heart disease (CHD) is one of the most common congenital malformations in infants and accounts for nearly one-third of significant congenital disabilities globally, which significantly burdens society and the economy. Such birth defects occur in the fetus while it is developing in the uterus during pregnancy. Fetal echocardiography is currently the most prevalent approach for CHD diagnosis. However, the existing prenatal diagnosis technology has certain limitations on the detection of CHD with late onset and insignificant early symptoms. Furthermore, the accuracy of the detection results is highly dependent on the quality of equipment and the detection skill of doctors.

An inherited predisposition of CHD with a high incidence of recurrence, along with the well-known association between CHD and chromosomal abnormalities points to a genetic foundation for the condition. Mutations in genes encoding transcription factors, cell signal transducers, and chromatin modifications can all disrupt the differentiation and arrangement of cell types necessary for heart development, causing defects in the structure and function of the heart. It is estimated that approximately 400 genes are linked to the onset of CHD. Since many of the proteins encoded by these genes collaborate and are interconnected through functional networks, a wide range of interaction networks might link to disorders.

Therefore, there is a need in the art for a non-invasive, efficient, and high-accuracy early CHD screening technology; the present invention addresses this need.

### SUMMARY OF THE INVENTION

It is an objective of the present invention to provide methods to solve the aforementioned technical problems.

The present invention identifies specific DNA structural variants (SVs) as reliable indicators of CHD. These SVs represent alterations in DNA sequences involving segments larger than 1,000 base pairs. They can include deletions, duplications, insertions, inversions, or translocations of genetic material. The presence of these SVs plays a pivotal role in predicting CHD, contributing to the genetic diversity of individuals and potentially leading to genetic disorders and diseases.

In accordance with a first aspect of the present invention, a method for detecting CHD in a fetus is provided. Particularly, the method includes the following steps:
obtaining a blood sample from a pregnant female;
extracting a fetal genomic DNA from the maternal blood sample;
measuring a level of the fetal genomic DNA for a panel of CHD-susceptible structural variants selected from a head-to-tail deletion or a tail-to-head duplication occurring on human chromosome 5, 6 or 15;
applying each of the measured fetal genomic DNA of the panel of CHD-susceptible structural variants against a database created by analyzing measured fetal genomic DNA levels of control subjects with no CHD, in which the applying compares the expression level for each of the CHD-susceptible structural variants to fetal genomic DNA levels of control subjects using whole-genome sequencing and the database includes a threshold value for the expression level for each of the CHD-susceptible structural variants; and
indicating that the pregnant female has an increased risk of expecting a fetus with CHD if the measured fetal genomic DNA of the panel of CHD-susceptible structural variants is greater than the threshold value.

In accordance with one embodiment of the present invention, the CHD-susceptible structural variants are specific markers are only expressed in a database created by analyzing measured fetal genomic DNA levels of CHD subjects and not presented in the database created by control subjects.

In accordance with one embodiment of the present invention, the CHD-susceptible SVs include a DEL-ht at 46,486,069-46,541,284 of chromosome 5 (chr5:g.46,486,069_46,541,284del), a DEL-ht at 51,874,769-51,880,809 of chromosome 6 (chr6:g.51,874,769-51,880,809del), a DUP-th at 46541462-46496661 of chromosome 5 (chr5:g.46541462-46496661dup) and a DUP-th at 17394590-17093403 of chromosome 15 (chr15:g.17394590-17093403dup).

In accordance with one embodiment of the present invention, the blood sample is obtained through non-invasive prenatal testing (NIPT).

In accordance with one embodiment of the present invention, the method further includes a risk assessment for CHD in the fetus based on the presence of the CHD-susceptible SVs, in which the risk assessment for CHD is performed using a rigorous and precise algorithm based on a dataset of CHD cases and controls.

In accordance with one embodiment of the present invention, the method further includes analyzing fetal genomic DNA from maternal sources to assess the genetic risk of CHD in the fetus.

In accordance with one embodiment of the present invention, the risk assessment is performed in conjunction with other prenatal diagnostic tests, such as fetal echocardiography and maternal serum screening.

### BRIEF DESCRIPTION OF THE DRAWINGS

Embodiments of the invention are described in more details hereinafter with reference to the drawings, in which:

FIGS. 1A-1B depicts a schematic diagram illustrating a CHD-susceptible structural variants analysis process in accordance with one embodiment of the present invention, in which FIG. 1A depicts the data acquisition and processing part and variants calling part, and FIG. 1B depicts the variant filtering and association analysis;

FIG. 2 shows the associated SV breakpoints between the unaffected control and CHD proband groups, in which only SV breakpoints that occur in >5% of the proband group are shown;

FIGS. 3A-3D depicts the evaluations of CHD-associated SVs between CHD proband group and unaffected control group, in which FIG. 3A depicts volcano plots showing the negative logarithm of the Benjamini-Hochberg false discovery rate test (q-value) and log₂ (Pro/Ctrl) of each SV breakpoint in CHD probands compared with the unaffected controls, FIG. 3B depicts volcano plots demonstrating a significant difference between the CHD proband group and the unaffected control group with chromosomal intervals of 100 bp, FIG. 3C displays the profile of different SV types of candidate SVs with variant frequency in the CHD probands, and FIG. 3D depicts the profile of different SV types of candidate SVs with variant frequency in the relative groups (parents of CHD probands); and

FIG. 4 shows the associated SV breakpoints between the relative and proband groups, in which only SV breakpoints that occur in >5% of the proband group are shown.

### DETAILED DESCRIPTION

In the following description, specific methods of prenatally diagnosing congenital heart disease (CHD) in a fetus are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

The present inventors have determined that the presence of particular types of DNA structural variants are predictors of congenital heart disease. DNA structural variants (SVs) are alterations in the DNA sequence that involve segments of DNA that are larger than 1,000 base pairs. These variations can involve deletions, duplications, insertions, inversions, or translocations of genetic material, and they play a significant role in the genetic diversity of individuals. SVs can affect the structure and function of genes, potentially leading to various genetic disorders and diseases.

Through the identification of these particular structural variants, the present invention provides a non-invasive and accurate screening technique for fetal CHD to the presence of these structural variants. In particular, the present invention can detect CHD in the fetus at around 8 weeks by means of genetic sequence detection.

In the method of the present invention for prenatal diagnosis of CHD in a developing fetus, a series of steps are performed to that result in both accurate results and early detection. This approach offers an assessment to expectant mothers, allowing them to proactively address potential fetal health concerns.

The process begins with the collection of a blood sample from the pregnant subject. This non-invasive procedure eases any concerns associated with prenatal testing, enhancing accessibility, and reducing the potential risks to both the mother and the developing fetus. This blood sample is a key source for the extraction of fetal genomic DNA, which contains essential genetic information about the developing fetus.

From this maternal blood sample, fetal genomic DNA is meticulously extracted. In general, the maternal blood sample is processed to isolate cell-free DNA. This can involve centrifugation or other methods to separate the plasma or serum from the blood cells. The cell-free DNA includes both maternal and fetal DNA fragments. Various techniques can be used to enrich the concentration of fetal DNA in the sample. This can involve methods such as size-based separation, methylation-based approaches, or targeted amplification of specific fetal DNA sequences. These methods help distinguish and isolate the relatively small portion of fetal DNA from the much larger amount of maternal DNA. Once the fetal DNA is enriched, genomic DNA extraction techniques can be used to isolate and purify the DNA from the rest of the sample components.

The fetal genetic material carries invaluable information about the fetus's genetic makeup, including potential markers associated with CHD. Whole-genome sequencing is performed on this fetal genomic DNA, enabling a comprehensive and detailed examination of the genetic data.

Within this vast genomic dataset, the method of the present invention focuses on specific markers proposed by the present invention and named as CHD-susceptible structural variants. These variants, such as head-to-tail deletions and tail-to-head duplications on human chromosomes 5, 6, or 15, have been identified as significant indicators of potential congenital heart disease. Their presence or absence is carefully scrutinized to determine the likelihood of CHD development.

Head-to-tail deletions are a type of structural variant in which a segment of DNA is deleted from one end to the other, resulting in the loss of genetic material. This can lead to the loss of one or more genes, regulatory elements, or other important functional sequences. These deletions can have various consequences, depending on the specific genes or regulatory elements that are affected. They can cause genetic disorders, predisposition to certain diseases, or other phenotypic changes.

Tail-to-head duplications involve the replication of a segment of DNA in the tail-to-head direction, leading to the presence of additional copies of the genetic material. This can result in an increased dosage of specific genes or regulatory elements, which can have various effects on the phenotype. Duplications can lead to gene overexpression, altered gene regulation, or an increased predisposition to certain genetic disorders or diseases.

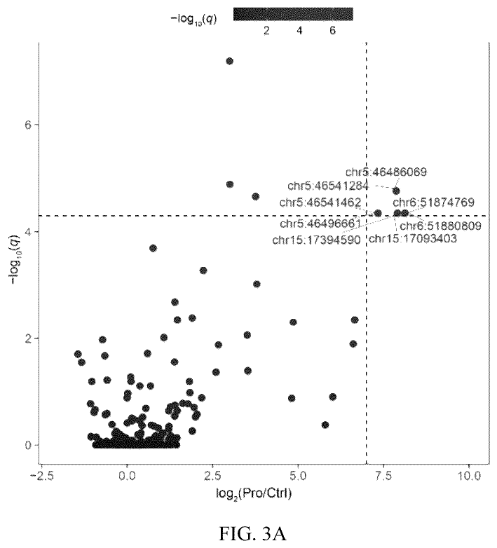

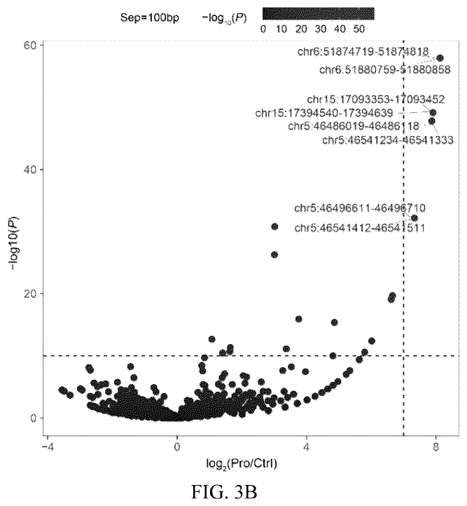

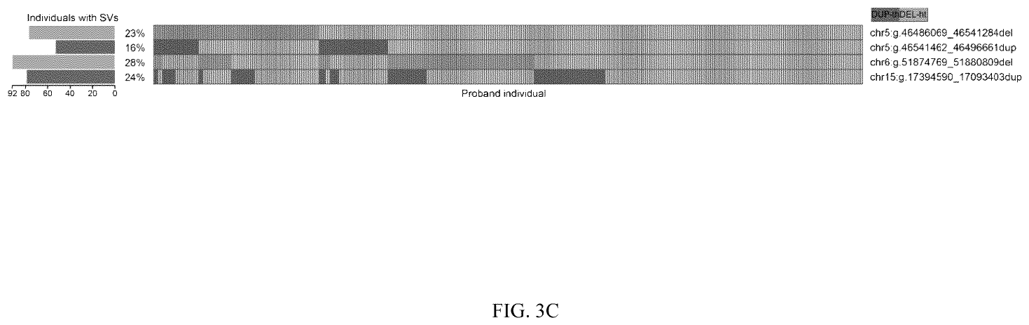

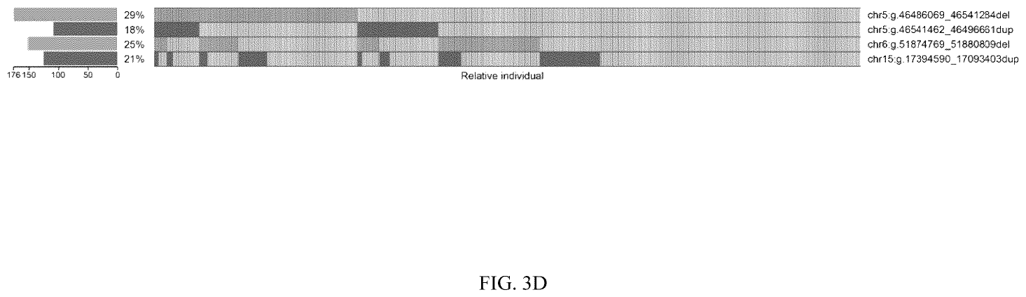

Both of these structural variants can arise spontaneously or can be inherited, and they can contribute to genetic diversity and evolution. However, when these variants occur within critical genomic regions, they can lead to various genetic disorders, developmental abnormalities, or an increased predisposition to certain diseases. As will be discussed in further detail, below, the present inventors determined that at least four susceptible SVs are related to CHD. These are identified in FIG. 3C. They include two deletions (chr5: g.46,486,069_46,541,284del and chr6: g.51,874,769_51,880,809del) and two duplications (chr5: g.46541462_46496661dup and chr15: g.17394590_17093403dup), covering a total of 63.6% of all CHD probands. These specific locations enhance the precision of the CHD detection process.

The measured fetal genomic DNA for these structural variants is then subjected to a comparative analysis. This analysis is carried out against a comprehensive database created by meticulously examining the fetal genomic DNA levels of control subjects who have been confirmed not to have CHD.

To ensure the highest level of accuracy and precision, this comparative analysis utilizes advanced whole-genome sequencing techniques. The method entails a sophisticated data comparison process that involves evaluating the expression level of each CHD-susceptible structural variant. This evaluation is conducted in relation to fetal genomic DNA levels of control subjects.

The database developed for this method encompasses threshold values for each of the CHD-susceptible structural variants. If the measured fetal genomic DNA levels for these variants surpass these threshold values, it serves as an indicator that the pregnant individual may have an increased risk of bearing a fetus with CHD.

Remarkably, the CHD-susceptible structural variants used in this method are unique markers. These markers are exclusive to the database generated through the analysis of fetal genomic DNA levels of individuals with CHD and are not found in the database of control subjects.

Through the detection of various structural DNA variants, the present invention provides a risk assessment for CHD in the developing fetus. This assessment is not just based on a singular marker but is enriched by further analysis, as set forth in the examples, regarding familial CHD and DNA structural variants

To provide a more holistic view of CHD risk, maternal genomic DNA is also analyzed. This additional step assesses the genetic risk of CHD in the fetus, considering both maternal and fetal genetic factors. By combining these insights, a comprehensive risk profile is constructed, allowing expectant parents and healthcare providers to make informed decisions.

The method also can further complement existing prenatal diagnostic tests. By working in tandem with fetal echocardiography and maternal serum screening, it enhances the accuracy and comprehensiveness of CHD diagnosis. This integrated approach further confirms the presence or absence of CHD, enabling parents and their medical team to take proactive measures for the health and well-being of their developing child.

The primary goal of early diagnosis is to facilitate timely intervention, thereby enhancing the prognosis for the fetus. In cases where there is a strong suspicion of CHD in the fetus, various interventions become possible, including general, interventional, and surgical treatments. More specifically, maternal intervention may involve administering oxygen as necessary to enhance fetal oxygen levels in utero. In certain situations, fetoscopy might be employed to improve the prospects of postnatal cardiac repair. Furthermore, early diagnosis serves as a foundational step for preparing surgical interventions after the birth of the child, enabling the medical team to make necessary preoperative preparations as soon as possible.

The use of the inventive can therefore enable a prenatal diagnosis, offering early and accurate detection of congenital heart disease in developing fetuses. It combines non-invasive sample collection, whole-genome sequencing, and genomic analysis-driven risk assessment to provide expectant parents and healthcare providers with information on fetal CHD.

### EXAMPLES

### Example 1. A Comprehensive Method for Detecting SVs Using Whole-Genome Sequencing Data

The present invention develops a pipeline for the accurate detection of SVs based on whole-genome sequencing (WGS) using cohort study and fusion algorithms. Briefly, the genomic DNA is extracted from venous blood samples of subjects, followed by WGS sequencing utilizing 150 base-pair paired reads, ensuring comprehensive coverage with a median depth exceeding 30 times per individual. High-quality sequence is achieved using state-of-the-art gene sequencers.

WGS data is mapped using specialized mapping software capable of efficiently aligning low-divergent sequences to a vast reference genome. The paired reads are meticulously mapped to a human reference genome (GRCh38). A set of utilities designed for interacting with high-throughput sequencing data is employed for indexing the alignments. Furthermore, a collection of command line tools is utilized to eliminate duplicate reads, ensuing the accuracy of subsequent SV detection.

The detection of SVs is executed using well-established methods and software tools, such as SvABA and Manta. These tools are renowned for their capability to identify SVs within sequencing data through genome-wide local assembly. To refine the pool of detected SVs, a filtering process is applied based on predefined detection thresholds. Only SVs meeting these criteria are retained as candidates. Additionally, the final set of SVs is determined by identifying the intersection of SVs called by multiple software.

SCs are classified based on four primary types: deletion (DEL-ht), duplication (DUP-th), inversion (INV-hh, INV-tt), and translocation (TRX-ht, TRX-th, TRX-hh, TRX-tt). To ensure the utmost precision, If SV breakpoints overlap within 100 bp at both the 5′ and 3′ ends and the SV types are concordant, they are considered the same SV. Meanwhile, to ensure the utmost precision, only SVs with a substantial number of supportive reads, totaling 20, and a minimum of 6 split reads are retained for further analysis.

### Example 2. Identifying a Series of Susceptible SVs Associated with CHD

In order to obtain accurate conclusions, the WGS data (with a median depth of >30×) is obtained from the Pediatric Cardiac Genomics Consortium (PCGC) for 330 CHD probands as the proband group, and their 612 parents as the relative group, respectively. The proband group includes isolated heart malformations and extracardiac anomalies, and it has no loss-of-function coding variants identified in whole-exome sequencing (WES) studies. In addition, WGS of 923 individuals, presumed unaffected from the 1,000 Genomes Project, serves as the control group.

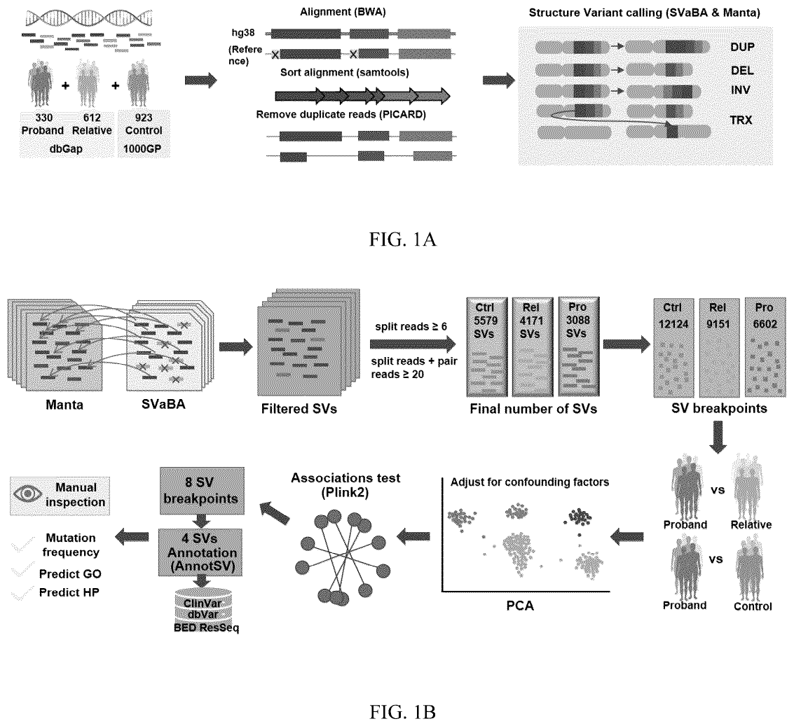

Briefly, as shown in FIG. 1A, the WGS data of CHD probands (n=330) and their relatives (n=612) and unaffected controls (n=923) are downloaded from dbGaP (Accession numbers: phs001138.v3.p2 and phs001194.v2.p2) and 1000GP, respectively. The reads are aligned using mapping software (Burrows-Wheeler-Alignment) and sorted and indexed by a set of utilities, such as samtools. Duplicate reads are removed by command line tools, such as PICARD. SvABA and Manta are applied to call SV, and only high-quality variants, which are flagged as “PASS”, are retained for analyses. Referring to FIG. 1B, the SV calls generated by SvABA and Manta are intersected while only retaining SVs with the total number of supported reads≥20 and split reads≥6, and association analyses for each SV breakpoint are performed using a logistic regression model after principal component analysis.

The obtained SVs of each sample are collected for further analysis. Each SV has two SV breakpoints, and SVs can share breakpoints. The detected SVs contributed 6,602, 9,151, and 12,124 SV breakpoints in the probands, the relatives, and the controls, respectively (FIG. 1B). Subsequently, the association analysis is applied to the SV breakpoints. CHD relatives and unaffected controls are defined as reference groups, respectively. Plink2 software is used for quality control and genetic association analysis, and the breakpoints that less than 5% of the proband group are filtered out for subsequent analyzes. Each SV breakpoint is tested for association using logistic regression models under principal component analysis (PCA) and identified by the Benjamini-Hochberg false discovery rate (q-value) to calculate multiple hypothesis tests.

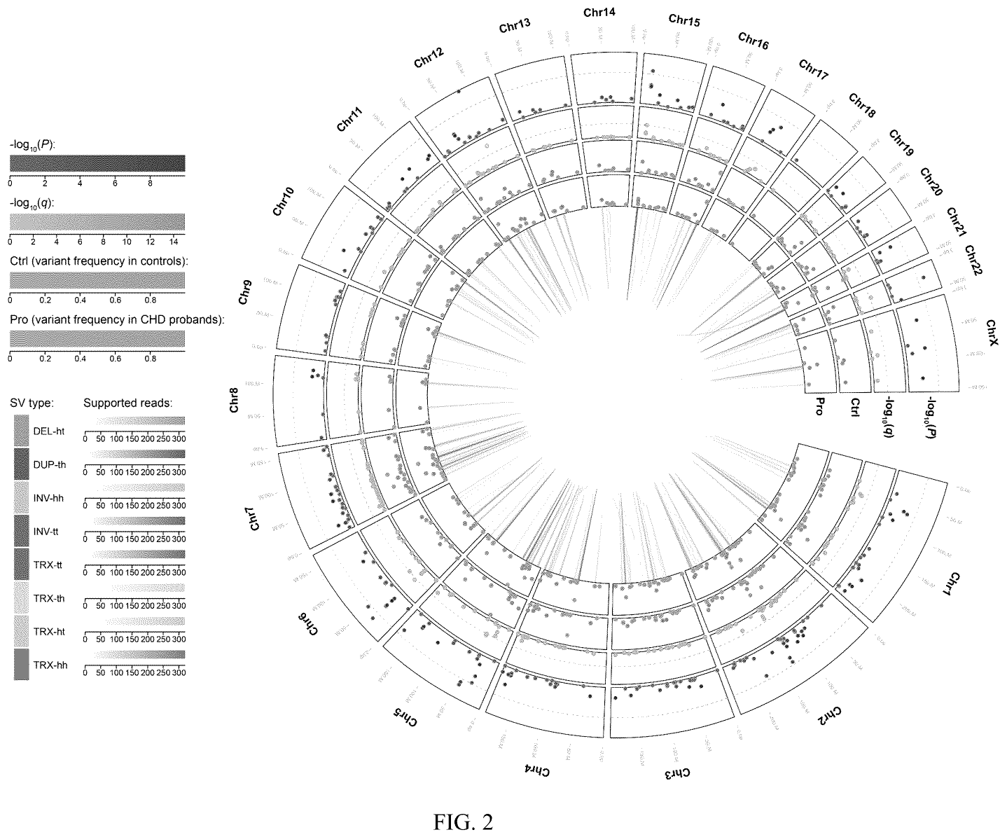

As shown in FIG. 2, compared with the unaffected control group, 13 independent SV breakpoints associated with CHD (P-value<1×10⁻⁶, Benjamini-Hochberg false discovery rate test (q-value<5×10⁻⁵) are identified. The variant frequency of the SV breakpoints in CHD probands and the unaffected controls are calculated, and the eight breakpoints with significant differences are detected, which means null enrichment appears in the unaffected controls at these breakpoints (FIG. 3A).

Following this lead, statistical analysis of the SV breakpoints according to the chromosome region window is performed for further validation. It is found that the control group does not have variants within 500 bp of the above-screened breakpoints. As shown in FIG. 3B, specifically, the SV breakpoints screened above are significantly different within 100-, 200-, and 500-bp windows in the CHD probands compared to the unaffected controls (Fisher's exact test P-value<1×10⁻¹⁰). The above results suggest that the eight SV breakpoints are hotspots related to CHD. Based on the above eight SV breakpoints associated with CHD, four susceptible SVs related to CHD are identified (FIG. 3C), with two deletions (chr5: g.46,486,069_46,541,284del and chr6: g.51,874,769_51,880,809del) and two duplications (chr5: g.46541462_46496661dup and chr15: g.17394590_17093403dup), covering a total of 63.6% of all CHD probands.

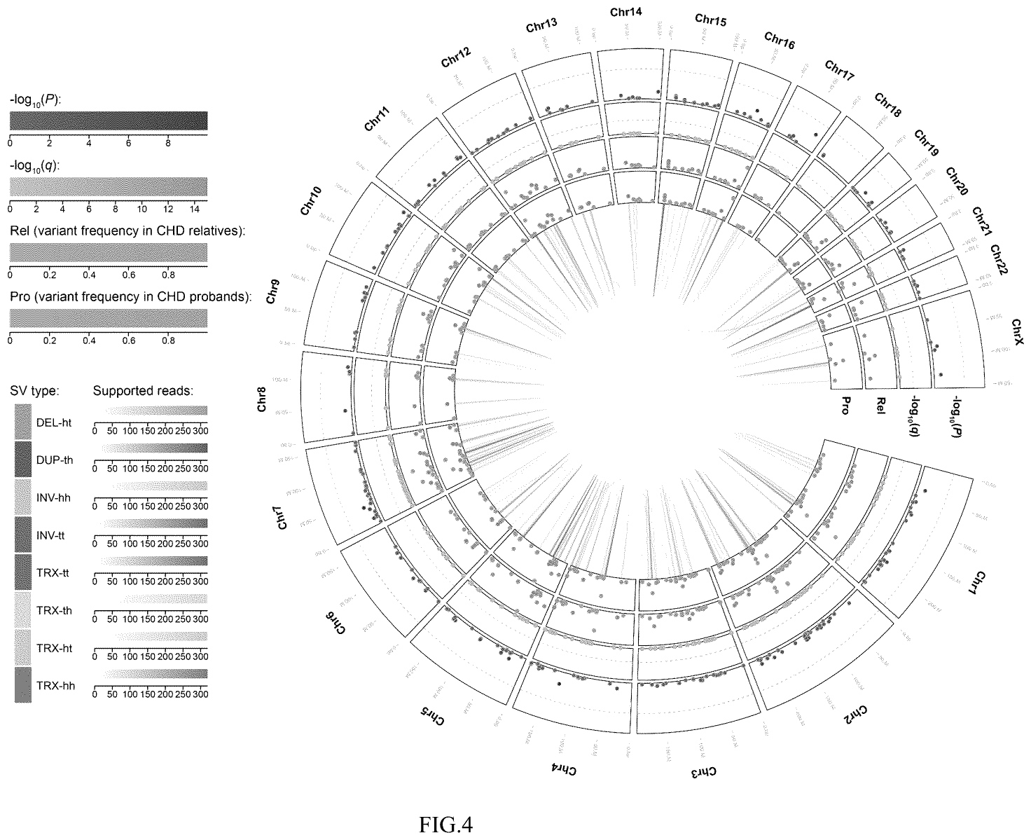

Moreover, the proband and relative groups are also compared. The comparison results reveal that there is no significant difference in SV breakpoints between these two groups (FIG. 4). The SV breakpoints at different sizes of chromosome bins are also examined, showing that no significant region within the range of 5 kbp (Fisher's exact test P>1×10⁻¹⁰). Next, the enrichment of the above four established CHD-susceptible SVs in the relative group are explored, and it is observed that the proportions of these SVs in the relative group are similar to those in the proband group (FIG. 3D). Then, the probands with these SVs are matched with their parents (proband-parent trios) for further study. Among the trios for which complete pedigree information is available, the parents of CHD probands with these four SVs are not necessarily identified as these SVs. It can be speculated that for one or both parents with these susceptible SVs, they might be passed on directly to the offspring. For both parents without susceptible SVs, children acquired these SVs de novo. In the 330 CHD cohort, it is observed that one identical de novo SV related to CHD recurred 24-43 times. According to the mean mutation rate of deletion (0.146) and duplication (0.040), every 7 or 25 live births have roughly one new deletion or duplication, respectively. Therefore, the acquisition of these de novo SVs due to fortuitous factors such as external damage can be ruled out. In other words, the susceptible SVs associated with CHD that are pinpointed are reliable.

### Example 3. The Detection of the WGS-Based Multi-Group Genetic Susceptibility SVs

Briefly, to practice the aforementioned method, 10 ml of venous blood is collected from pregnant women around 8 weeks of pregnancy in an anticoagulant tube containing EDTA. All samples are stored at −80° C. for genomic DNA extraction. The DNA of the fetus is isolated from the venous blood of the pregnant woman, sequencing with 150-bp paired reads and a median depth of more than 30 per individual is next performed by Illumina HiSeq 2000. And then, follow the protocol proposed above to obtain the SVs of the fetuses. As four susceptible SVs related to CHD cover a total of 63.6% of all CHD probands, the detection technology can accurately diagnose more than 60% of CHD. As long as the above-mentioned susceptible SVs are detected in the fetuses, it is highly suggestive of the occurrence of CHD, and thus prompts the need for early intervention.

In conclusion, the present invention provides a detection approach for screening neonatal CHD, and a non-invasive, efficient, and accurate early CHD screening protocol. In short, the CHD screening protocol can be used by users, such as medical institutions, to process and test blood samples from pregnant women to determine whether the fetuses have CHD.

Compared with the traditional prenatal diagnosis of CHD, the present invention has many advantages, such as early discovery, low cost, and simple operation. The present invention advances the CHD detection period from 18 to 24 weeks in the traditional technology, to around 8 weeks, which is of great significance for CHD, a disease with a very high early cure rate. Compared with the traditional detection approach, the present invention reduces the dependence on medical equipment and reduces the cost of medical equipment. Different from the traditional detection approach, which has higher requirements on the quality of medical equipment and professional level of medical faculty, the detection approach of the present invention is simple to use, basically eliminates human errors, and can be used for user self-testing, providing self-testing methods, and optimizing medical configuration.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A method for detecting congenital heart disease (CHD) in a fetus, comprising:
obtaining a blood sample from a pregnant female;
extracting a fetal genomic DNA from the maternal blood sample;
measuring a level of the fetal genomic DNA for a panel of CHD-susceptible structural variants selected from a head-to-tail deletion or a tail-to-head duplication occurring on human chromosome 5, 6 or 15;
applying each of the measured fetal genomic DNA of the panel of CHD-susceptible structural variants against a database created by analyzing measured fetal genomic DNA levels of control subjects with no CHD;
wherein the applying compares the expression level for each of the CHD-susceptible structural variants to fetal genomic DNA levels of control subjects using whole-genome sequencing, wherein the database comprises a threshold value for the expression level for each of the CHD-susceptible structural variants; and
indicating that the pregnant female has an increased risk of expecting a fetus with CHD if the measured fetal genomic DNA of the panel of CHD-susceptible structural variants is greater than the threshold value.

2. The method of claim 1, wherein the CHD-susceptible structural variants are specific markers are only expressed in a database created by analyzing measured fetal genomic DNA levels of CHD subjects and not presented in the database created by control subjects.

3. The method of claim 1, wherein the CHD-susceptible structural variants comprise a head-to-tail deletion at 46,486,069-46,541,284 of chromosome 5 (chr5:g.46,486,069_46,541,284del), a head-to-tail deletion at 51,874,769-51,880,809 of chromosome 6 (chr6:g.51,874,769-51,880,809del), a tail-to-head duplication at 46541462-46496661 of chromosome 5 (chr5:g.46541462-46496661dup) and a tail-to-head duplication at 17394590-17093403 of chromosome 15 (chr15:g.17394590-17093403dup).

4. The method of claim 1, wherein the blood sample is obtained through non-invasive prenatal testing (NIPT).

5. The method of claim 1, wherein the method further comprises a risk assessment for CHD in the fetus based on the presence of the CHD-susceptible structural variants, wherein the risk assessment for CHD is performed using a rigorous and precise algorithm based on a dataset of CHD cases and controls.

6. The method of claim 1, wherein the method further comprises analyzing maternal genomic DNA to assess the genetic risk of CHD in the fetus.

7. The method of claim 5, wherein the risk assessment is performed in conjunction with other prenatal diagnostic tests, comprising fetal echocardiography and maternal serum screening.
